(19) United States
(12) Patent Application Publication
(10) Pub. No.: US 2020/0357489 A1
(43) Pub. Date: Nov. 12, 2020

(54) DEEP LEARNING-BASED QUICK AND PRECISE HIGH-THROUGHPUT DRUG SCREENING SYSTEM

(71) Applicant: Shanghai Tongji Hospital, Putuo District Shanghai (CN)

(72) Inventors: Liming Cheng, Shanghai (CN); Rongrong Zhu, Shanghai (CN); Yanjing Zhu, Shanghai (CN)

(21) Appl. No.: 16/962,313

(22) PCT Filed: Nov. 30, 2018
(86) PCT No.: PCT/CN2018/118397

§ 371 Date: Jul. 15, 2020

(30) Foreign Application Priority Data
Jan. 23, 2018 (CN) 201810063786.X

(51) Int. Cl. G16C 20/70; G16C 20/64; G06N 3/08; G06K 9/62

(52) U.S. Cl. CPC G16C 20/70; G06K 9/628; G06N 3/08; G16C 20/64

(57) ABSTRACT

A deep learning-based quick and precise high-throughput drug screening system, comprising a picture preprocessing module and a neural network module. The picture preprocessing module comprises a channel merging module and a picture standardization module. The channel merging module merges different cell single-color channel pictures into a multi-channel picture representation, and the tensor of the picture obtained after the merging is represented as [H,W,C]; the picture standardization module standardizes input multi-channel picture data into the tensor representation of [70,70,C]; the neural network module functions subsequent to the picture standardization module, the input data of the neural network module is the tensor of the standardized picture, and final predictive classification determination is implemented by the trained neural network. The established deep learning-based drug screening system DeepScreen has the advantages of high throughput, precision, high efficiency, high speed, convenience, low costs and interference resistance, and has a practical application prospect worth concerning.

### TECHNICAL FIELD OF THE INVENTION

The present invention refers to the technical field of biomedicine and artificial intelligence, particularly to a deep learning-based quick and precise high-throughput drug screening system.

### BACKGROUND OF THE INVENTION

According to statistics, it takes 10-14 years for each new drug from research, testing to product launch, costing more than US$200 million. How to speed up the discovery and testing of new drugs has always been the key and difficult point of accelerating drug development. In recent years, the development of disciplines such as biochemistry, physiology and pathology has led to new methods for drug screening, such as some molecular and cellular drug screening models. High throughput screening technology (HTS) was developed in the late 1990s based on the described models and the development of more advanced detection technology, automation technology and computer technology. HTS relies mainly on automated operating systems, ie laboratory robot and high-sensitivity detection methods, including spectrophotometry and fluorescence detection technology. The emergence of HTS has greatly accelerated the speed of drug screening, but it still has great limitations, including high cost, difficulty in model establishment, and limited number of models. China started late in the study of drug screening systems, and only a few national key laboratories have HTS systems. Laboratory robots are difficult to popularize due to their high cost, and various detection methods are still inseparable from manual statistics and analysis.

To the present, new drug screening technology has been increasingly combined with rapidly improving computer technology. In previous studies, computer technology was mostly used for the statistical processing of experimental data and the analysis and classification of existing features. Further applications of computer technology include computer-aided drug design. In recent years, there have been some studies applying machine learning to improve the effect of virtual screening. However, although virtual screening plays an important role in drug screening, it still relies on the existing small molecule database and various features that have been artificially classified, which is not enough to reflect the actual application efficacy of drugs. Various scientific research institutions and laboratories need a new drug screening system that can be used to evaluate drug efficacy. The drug screening system must have the advantages of high accuracy, strong anti-interference ability, short time consumption and low costs rather than the high cost like laboratory robots, and not limited by the existing database and artificial feature classification.

In summary, the existing drug screening system cannot meet the growing scientific research needs. Therefore, it is critical to establish a simple, efficient, accurate, and low-cost high-throughput drug screening system. We consider applying machine learning methods to the establishment of laboratory drug screening systems.

Deep learning is a branch of machine learning. Its concept is derived from the research of artificial neural networks. According to imitating the mechanism of the human brain to observe and interpret various data, it combines low-level features to form high-level representation attribute categories, thereby discovering distributed characteristics of data. Deep learning has become a research hotspot in the field of artificial intelligence in recent years because that its training process can extract and integrate features, collect and process large data, and has excellent universal applicability.

Chinese Patent Application No. 2017101273955 disclosed an intelligent lead compound discovery method based on convolutional neural network, which solves the problems of low efficiency and low accuracy of current lead compound virtual screening. In this method, first, the structural formula of the compound is transformed into a plane picture, and the black-and-white and anti color processing are carried out. Then all the pictures are classified according to the active attributes of the compounds, labeled with numbers according to the categories, and input into the system. Next, a part of the pictures are selected as training set for the convolutional neural network to learn how to classify deeply, and the rest of the pictures are used as test set to evaluate the model. Finally, after the deep learning is completed, a same processed picture other than the training set and the test set is input to the system to calculate the probability that the compound on the picture has a certain active attribute.

However, the deep learning-based quick and precise high-throughput drug screening system of the present invention has not been reported so far.

### SUMMARY OF THE INVENTION

In the present invention, a deep learning-based quick and precise high-throughput drug screening system is established, using deep learning method to train data for the first time. The system has the advantages of high accuracy, high efficiency, high speed and anti-interference, which greatly shortens the time to judge the drug efficacy, and is expected to replace the existing experimental methods drug evaluation.

In one aspect, the present invention provides a deep learning-based quick and precise high-throughput drug screening system, said deep learning-based quick and precise high-throughput drug screening system comprises a picture preprocessing module and a neural network module; said picture preprocessing module comprising a channel merging module and a picture standardization module; the input data of said channel merging module are single-color channel pictures of cells, said channel merging module merges different cell single-color channel pictures into a multi-channel picture representation, and the tensor of the picture obtained after the merging is represented as [H,W,C]; said picture standardization module functions subsequent to said channel merging module, the input data of said picture standardization module is the tensor of merged picture, said picture standardization module standardizes the input multi-channel picture data into the tensor representation of [70,70,C], the standardization method is as follows:

1) use bicubic interpolation algorithm to transform the picture tensor of [H, W, C] to [70, 70, C];

2) treat the picture tensor after interpolation by regularization method;

said neural network module functions subsequent to the picture standardization module, the input data of the neural network module is the tensor of the standardized picture, and final predictive classification determination is obtained by the trained neural network. Said predictive classification determination is as follows:

| label | description |
|---|---|
| 0 | No efficacy |
| 1 | Low efficacy |
| 2 | Moderate efficacy |
| 3 | High efficacy |

In a preferred embodiment, the network structure of said neural network module is as follows:

| | convolution kernel (number) size/ |
|---|---|
| Types | stride (or notes) |
| convolution | (32) 3 × 3/1 |
| convolution | (64) 3 × 3/1 |
| convolution | (80) 1 × 1/1 |
| convolution | (192) 3 × 3/1 |
| pooling | (−) 3 × 3/2 |
| Module 1 | 3 × sub-network module 1 |
| Module 2 | 5 × sub-network module 2 |
| Module 3 | 3 × sub-network module 3 |
| pooling | (−) 8 × 8/1 |
| convolution | (4) 1 × 1/1 |
| Softmax | classification output |

More preferably, the network structure of said sub-network module 1 is as follows:

input (Input goes into each branch)

| branch 1 | branch 2 | branch 3 | branch 4 |
|---|---|---|---|
| convolution | convolution | convolution | pooling (−) |
| (64) 1 × 1/1 | (48) 1 × 1/1 | (64) 1 × 1/1 | 3 × 3/1 |
| | | convolution | |
| | | (96) 3 × 3/1 | |
| | convolution | convolution | convolution |
| | (64) 5 × 5/1 | (96) 3 × 3/1 | (64) 1 × 1/1 |

Merge 4 branches along the channel direction

More preferably, the network structure of said sub-network module 2 is as follows:

input (Input goes into each branch)

| branch 1 | branch 2 | branch 3 | branch 4 |
|---|---|---|---|
| convolution | convolution | convolution | pooling (−) |
| (192) 1 × 1/1 | (128) 1 × 1/1 | (128) 1 × 1/1 | 3 × 3/1 |
| | | convolution | |
| | | (128) 7 × 1/1 | |
| | convolution | convolution | |
| | (128) 1 × 7/1 | (128) 1 × 7/1 | |
| | | convolution | convolution |
| | | (128) 7 × 1/1 | (192) 1 × 1/1 |
| | convolution | convolution | |
| | (192) 7 × 1/1 | (192) 1 × 7/1 | |

Merge 4 branches along the channel direction

More preferably, the network structure of said sub-network module 3 is as follows:

input (Input goes into each branch)

| branch 1 | branch 2 | | branch 3 | | branch 4 |
|---|---|---|---|---|---|
| convolution | convolution | | convolution | | pooling (−) |
| (320) 1 × 1/1 | (384) 1 × 1/1 | | (448) 1 × 1/1 | | 3 × 3/1 |
| | branch | branch | convolution | | |
| | 2a | 2b | (384) 3 × 3/1 | | |
| | convolution | convolution | branch | branch | |
| | (384) 1 × 3/1 | (384) 3 × 1/1 | 3a | 3b | |

| | | | |
|---|---|---|---|
| Merge 2 branches along the | convolution | convolution | convolution |
| channel direction | (384) 1 × 3/1 | (384) 3 × 1/1 | (192) 1 × 1/1 |

Merge 2 branches along
the channel direction

Merge 4 branches along the channel direction

In another preferred embodiment, the training method of neural network is as follows: train neural network on two NVIDIA GTX 1080ti video cards by using TensorFlow framework; use Adam optimizer as training optimizer and determine the corresponding training parameters as: learning rate 0.001, beta1 0.9, beta2 0.999, epsilon 1e-8.

In another aspect, the present invention provides a method of deep learning-based quick and precise high-throughput drug screening, comprising:

1) treating A549 cells and HepG2 cells with traditional medicine and nano medicine for two hours and six hours respectively, then staining with fluorescent antibody and getting the cell picture;

2) inputting cell single-color channel pictures into a image preprocessing module to obtain standardized picture data;

3) importing standardized picture data into neural network module to obtain final predictive classification determination.

In a preferred embodiment, said picture preprocessing module comprising a channel merging module and a picture standardization module; the input data of said channel merging module is cell single-color channel pictures, said channel merging module merges different cell single-color channel pictures into a multi-channel picture representation, and the tensor of the picture obtained after the merging is represented as [H,W,C]; said picture standardization module functions subsequent to said channel merging module, the input data of said picture standardization module is the tensor of merged picture, said picture standardization module standardizes the input data into the tensor representation of [70,70,C], the standardization method is as follows:

1) use bicubic interpolation algorithm to transform the picture tensor of [H, W, C] to [70, 70, C];

2) treat the picture tensor after interpolation by regularization method;

said neural network module functions subsequent to the picture standardization module, the input data of the neural network module is the tensor of the standardized picture, and final predictive classification determination is obtained by the trained neural network.

In another preferred embodiment, said predictive classification determination is as follows:

| label | description |
|---|---|
| 0 | No efficacy |
| 1 | Low efficacy |
| 2 | Moderate efficacy |
| 3 | High efficacy |

In another preferred embodiment, the network structure of said neural network module is as follows:

| | convolution kernel (number) size/ |
|---|---|
| Types | stride (or notes) |
| convolution | (32) 3 × 3/1 |
| convolution | (64) 3 × 3/1 |
| convolution | (80) 1 × 1/1 |
| convolution | (192) 3 × 3/1 |
| pooling | (−) 3 × 3/2 |
| Module 1 | 3 × sub-network module 1 |
| Module 2 | 5 × sub-network module 2 |
| Module 3 | 3 × sub-network module 3 |
| pooling | (−) 8 × 8/1 |
| convolution | (4) 1 × 1/1 |
| Softmax | classification output |

More preferably, the network structure of said sub-network module 1 is as follows:

input (Input goes into each branch)

| branch 1 | branch 2 | branch 3 | branch 4 |
|---|---|---|---|
| convolution | convolution | convolution | pooling (−) |
| (64) 1 × 1/1 | (48) 1 × 1/1 | (64) 1 × 1/1 | 3 × 3/1 |
| | | convolution | |
| | | (96) 3 × 3/1 | |
| | convolution | convolution | convolution |
| | (64) 5 × 5/1 | (96) 3 × 3/1 | (64) 1 × 1/1 |

Merge 4 branches along the channel direction

More preferably, the network structure of said sub-network module 2 is as follows:

input (Input goes into each branch)

| branch 1 | branch 2 | branch 3 | branch 4 |
|---|---|---|---|
| convolution | convolution | convolution | pooling (−) |
| (192) 1 × 1/1 | (128) 1 × 1/1 | (128) 1 × 1/1 | 3 × 3/1 |
| | | convolution | |
| | | (128) 7 × 1/1 | |
| | convolution | convolution | |
| | (128) 1 × 7/1 | (128) 1 × 7/1 | |
| | | convolution | convolution |
| | | (128) 7 × 1/1 | (192) 1 × 1/1 |
| | convolution | convolution | |
| | (192) 7 × 1/1 | (192) 1 × 7/1 | |

Merge 4 branches along the channel direction

More preferably, the network structure of said sub-network module 3 is as follows:

input (Input goes into each branch)

| branch 1 | branch 2 | | branch 3 | | branch 4 |
|---|---|---|---|---|---|
| convolution | convolution | | convolution | | pooling (−) |
| (320) 1 × 1/1 | (384) 1 × 1/1 | | (448) 1 × 1/1 | | 3 × 3/1 |
| | branch | branch | convolution | | |
| | 2a | 2b | (384) 3 × 3/1 | | |
| | convolution | convolution | branch | branch | |
| | (384) 1 × 3/1 | (384) 3 × 1/ 1 | 3a | 3b | |

| | | | |
|---|---|---|---|
| Merge 2 branches along the | convolution | convolution | convolution |
| channel direction | (384) 1 × 3/1 | (384) 3 × 1/1 | (192) 1 × 1/1 |

Merge 2 branches along
the channel direction

Merge 4 branches along the channel direction

In another preferred embodiment, the training method of neural network is as follows: train neural network on two NVIDIA GTX 1080ti video cards by using TensorFlow framework; use Adam optimizer as training optimizer and determine the corresponding training parameters as: learning rate 0.001, beta1 0.9, beta2 0.999, epsilon 1e-8.

The advantages of this invention are as follows:

1. The drug screening models based on deep learning in the prior art are all virtual. However, the deep learning-based quick and precise high-throughput drug screening model in this invention can assess the true efficacy of drugs because the model is trained using experimental data.

2. The model has a very high test accuracy when used to predict the efficacy of conventional drugs and drugs loaded on drug loading systems, that is, the drug delivery system does not affect the judgment of the model.

3. This model can predict the efficacy of drugs with high accuracy, even the drug only acts on cells for 2 hours or 6 hours. Therefore, the consumption time is greatly shortened, but drug efficacy assessment by traditional MTT colorimetry or flow cytometry cannot be completed in such a short time.

4. The accuracy of drug efficacy assessment cannot be affected by the autofluorescence of the drug, but traditional methods may get wrong results because of misreading fluorescence data.

5. The model has a high accuracy in predicting drug efficacy, regardless of whether the inputted picture is related to antibody-stained cells or not. Of course, the picture of antibody-stained cells can increase its accuracy. So, Users can choose flexibly according to their needs.

6. In the model training data set, the fluorescent drug curcumin is involved, which enhances the anti-interference ability of the model.

7. Based on the convolutional neural network, the deep learning method is used to build the model, which avoids the assessment error caused by artificial screening features.

8. The input data is cell pictures, and the requirements for equipment are low. So, the construction costs and test costs of the system in this invention are very low.

### BRIEF DESCRIPTION OF THE DRAWINGS

FIG. 1 shows an example of training data for the neural network. Ch09 and Ch01 are white light channels, Ch11 is red fluorescent staining, Ch02 is green fluorescent channel. The left images show two fluorescently labeled antibodies staining of A549 group (Ch11, Ch02), and the right images show a fluorescence stain (red, ChM and curcumin spontaneous green fluorescence (Ch02) interference of HepG2 group.

FIG. 2 is a schematic diagram of the model training and testing process.

FIG. 3 shows the training data, test data, and accuracy of the model. K represents white light image data, R represents red channel image data, and G represents green channel image data.

### DETAILED DESCRIPTION OF THE PREFERRED EMBODIMENT

The invention is further illustrated below in conjunction with specific embodiments. These examples are for illustrative purposes only and are not intended as limitations on the scope of the invention. In addition, changes therein and other uses will occur to those skilled in the art. Such changes and other uses can be made without departing from the scope of the invention as set forth in the claims.

### Example 1 A Deep Learning-Based Quick and Precise High-Throughput Drug Screening System

In the present invention, the cell pictures are used as input data. After training based on Convolutional Neural Network (CNN), a classification model “DeepScreen” for drug efficiency judgment is generated. The model shows a very high accuracy in the test of drug efficiency. Thus, the problems of the existing high-throughput drug screening systems are solved.

The following describes the construction process of the drug screening system model.

The A549 cells and HepG2 cells were treated with traditional medicine and drugs loaded on drug loading systems for two hours and six hours respectively, then stained with fluorescent antibody and photographed to get the cell pictures of training set.

The DeepScreen model mainly includes two parts, the picture preprocessing module and the neural network module. The operation process is as follows:

1. input cell single-color channel pictures into the picture preprocessing module to obtain standardized picture data;

2. input the standardized picture data into the neural network module to obtain the final predictive classification determination.

The final predictive classification determination is as follows:

| label | description |
|---|---|
| 0 | No effcacy |
| 1 | Low effcacy |
| 2 | Moderate efficacy |
| 3 | High efficacy |

The picture preprocessing module comprises two sub-modules, a channel merging module and a picture standardization module.

1. The Channel Merging Module

The input data of the channel merging module is cell single-color channel pictures, and each color channel is derived from the corresponding cell coloring channel. These cell single-color channel pictures must have the same height H and width W. The channel merging module merges these cell single-color channel pictures into a multi-channel picture representation. If the number of color channels input at one time is C, the tensor of the picture obtained after the merging is represented as [H, W, C].

2. The Picture Standardization Module

The input data of the picture standardization module which functions subsequent to the channel merging module is the tensor of the picture obtained after the merging represented as [H, W, C]. Since the input data of different batches may have different heights H and widths W, the function of the picture standardization module is to standardize the input data into the tensor representation of [70,70,C]. The specific method is as follows:

1) use bicubic interpolation algorithm to transform the picture tensor of [H, W, C] to [70, 70, C];

2) treat the picture tensor after interpolation by regularization method.

The neural network module functions subsequent to the picture standardization module, the input data of the neural network module is the standardized tensor representation of [70,70,C], and final predictive classification determination is obtained by the trained neural network. Network structure is as follows:

| | convolution kernel (number) size/ |
|---|---|
| Types | stride (or notes) |
| convolution | (32) 3 × 3/1 |
| convolution | (64) 3 × 3/1 |
| convolution | (80) 1 × 1/1 |
| convolution | (192) 3 × 3/1 |
| pooling | (−) 3 × 3/2 |
| Module 1 | 3 × sub-network module 1 |
| Module 2 | 5 × sub-network module 2 |
| Module 3 | 3 × sub-network module 3 |
| pooling | (−) 8 × 8/1 |
| convolution | (4) 1 × 1/1 |
| Softmax | classification output |

The network structure of sub-network module 1 is as follows:

input (Input goes into each branch)

| branch 1 | branch 2 | branch 3 | branch 4 |
|---|---|---|---|
| convolution | convolution | convolution | pooling (−) |
| (64) 1 × 1/1 | (48) 1 × 1/1 | (64) 1 × 1/1 | 3 × 3/1 |
| | | convolution | |
| | | (96) 3 × 3/1 | |
| | convolution | convolution | convolution |
| | (64) 5 × 5/1 | (96) 3 × 3/1 | (64) 1 × 1/1 |

Merge 4 branches along the channel direction

The network structure of sub-network module 2 is as follows:

input (Input goes into each branch)

| branch 1 | branch 2 | branch 3 | branch 4 |
|---|---|---|---|
| convolution | convolution | convolution | pooling (−) |
| (192) 1 × 1/1 | (128) 1 × 1/1 | (128) 1 × 1/1 | 3 × 3/1 |
| | | convolution | |
| | | (128) 7 × 1/1 | |
| | convolution | convolution | |
| | (128) 1 × 7/1 | (128) 1 × 7/1 | |
| | | convolution | convolution |
| | | (128) 7 × 1/1 | (192) 1 × 1/1 |
| | convolution | convolution | |
| | (192) 7 × 1/1 | (192) 1 × 7/1 | |

Merge 4 branches along the channel direction

The network structure of sub-network module 3 is as follows:

input (Input goes into each branch)

| branch 1 | branch 2 | | branch 3 | | branch 4 |
|---|---|---|---|---|---|
| convolution | convolution | | convolution | | pooling (−) |
| (320) 1 × 1/1 | (384) 1 × 1/1 | | (448) 1 × 1/1 | | 3 × 3/1 |
| | branch | branch | convolution | | |
| | 2a | 2b | (384) 3 × 3/1 | | |
| | convolution | convolution | branch | branch | |
| | (384) 1 × 3/1 | (384) 3 × 1/1 | 3a | 3b | |

| | | | |
|---|---|---|---|
| Merge 2 branches along the | convolution | convolution | convolution |
| channel direction | (384) 1 × 3/1 | (384) 3 × 1/1 | (192) 1 × 1/1 |

Merge 2 branches along the
channel direction

Merge 4 branches along the channel direction

Training method: We used TensorFlow framework to train neural network on two NVIDIA GTX 1080ti video cards. The training optimizer is Adam optimizer, and the corresponding training parameters are: learning rate is 0.001, beta1 is 0.9, beta2 is 0.999, epsilon is 1e-8.

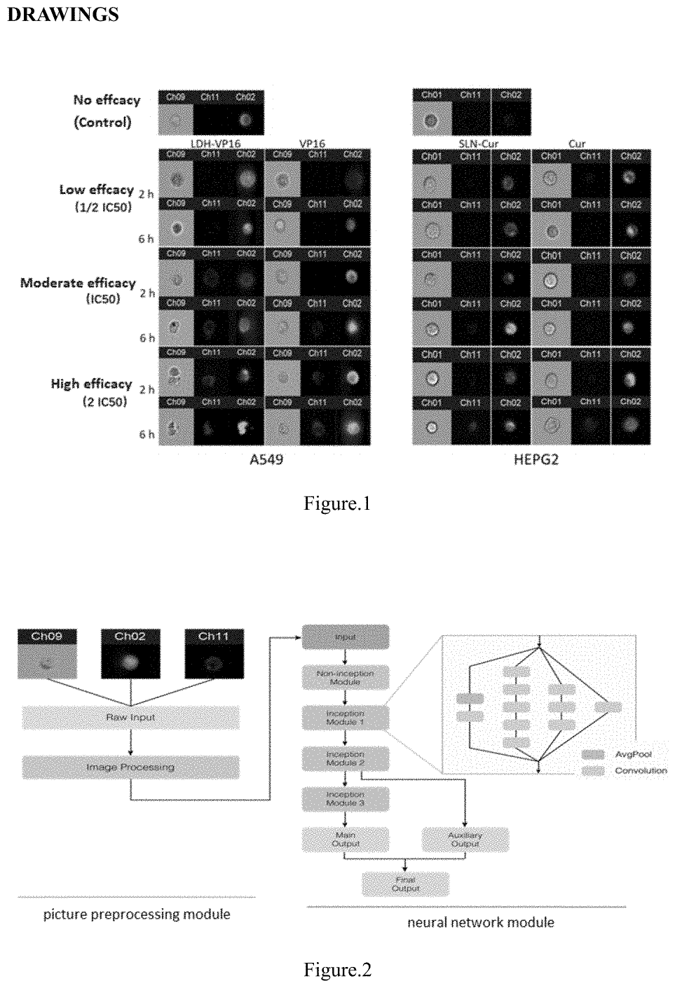

The classification model of the drug screening system constructed above was used to predict drug efficacy to evaluate the accuracy of the model. FIG. 1 shows an example of training data for the neural network. Ch09 and Ch01 are white light channels, Ch11 is red fluorescent staining, Ch02 is green fluorescent channel. The left image shows two fluorescently labeled antibodies staining of A549 group (Ch11, Ch02), and the right image shows a fluorescence stain (red, Ch11) and curcumin spontaneous green fluorescence (Ch02) interference of HepG2 group. The training benchmark settings are shown in the table below. The A549 cells and HepG2 cells were treated with traditional drugs or drugs loaded on drug loading systems that the efficacy are known to obtain the classification settings used for training. In the table below, LDH is a layered double hydroxide, VP16 is etoposide, SLN is lipid nanoparticles, and Cur is curcumin.

| Cell type | A549 | | HEpG2 | |
|---|---|---|---|---|
| No effcacy | non treatment | | non treatment | |
| Low effcacy | 10 μg/ml | 50 μg/ml | 3 μg/ml | 5 μg/ml |
| | LDH-VP16 | VP16 | SLN-Cur | Cur |
| Moderate | 20 μg/ml | 100 μg/ml | 6 μg/ml | 10 μg/ml |
| efficacy | LDH-VP16 | VP16 | SLN-Cur | Cur |
| High efficacy | 40 μg/ml | 200 μg/ml | 12 μg/ml | 20 μg/ml |
| | LDH-VP16 | VP16 | SLN-Cur | Cur |

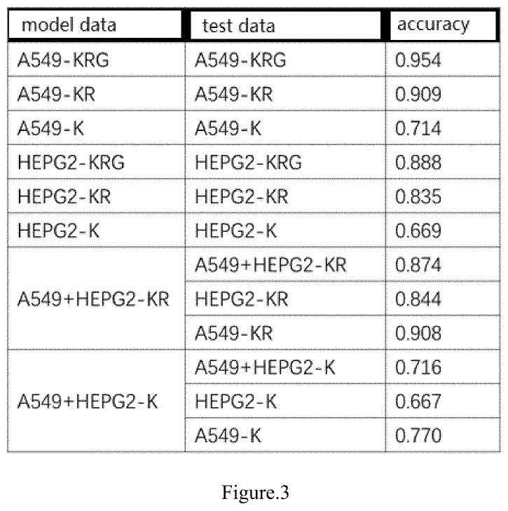

FIG. 2 is a schematic diagram of the model training and testing process. FIG. 3 shows the training data, test data, and accuracy of the model. K represents white light image data, R represents red channel image data, and G represents green channel image data in FIG. 3. Our research shows that DeepScreen model shows a very high accuracy in the test. Specifically, the accuracy of the model trained by pure white light cell image is as high as 0.7, the accuracy of the model trained by single fluorescent and white light image is as high as 0.87, and the accuracy of the model tested by double fluorescent antibody white light image training is as high as 0.95. Compared with the existing high-throughput virtual drug screening methods based on machine learning, the method of the present invention applies the advantages of machine learning without artificial feature mark to drug evaluation, thereby avoiding the influence of human subjective factors on drug evaluation. Compared with traditional laboratory drug evaluation methods, the DeepScreen model-based method of the present invention has the advantages of high throughput, high accuracy, short time consumption and low costs. In addition, we found that the model has a strong anti-interference ability for the evaluation of drugs with spontaneous fluorescence, and there is no significant difference in the accuracy of the model between drugs with or without fluorescence interference. In conclusion, DeepScreen, a drug screening system based on deep learning, has the advantages of high throughput, precision, high efficiency, high speed, convenience, low costs and interference resistance, and has a practical application prospect worthy of attention.

The methods described herein are presently representative of preferred embodiments. It will be apparent to one skilled in the art that various changes and modifications can be made therein without departing from the spirit and scope thereof. Such changes and modifications are intended to be encompassed by the scope of the following claims.

1. A deep learning-based quick and precise high-throughput drug screening system, wherein said deep learning-based quick and precise high-throughput drug screening system comprises a picture preprocessing module and a neural network module; said picture preprocessing module comprises a channel merging module and a picture standardization module; the input data of said channel merging module is cell single-color channel pictures, said channel merging module merges different cell single-color channel pictures into a multi-channel picture representation, and the tensor of the picture obtained after the merging is represented as [H,W,C]; said picture standardization module functions subsequent to said channel merging module, the input data of said picture standardization module is the tensor of merged picture, said picture standardization module standardize the input data into the tensor representation of [70,70,C], the standardization method is as follows:
1) use bicubic interpolation algorithm to transform the picture tensor of [H, W, C] to [70, 70, C];
2) treat the picture tensor after interpolation by regularization method;
said neural network module functions subsequent to the picture standardization module, the input data of the neural network module is the tensor of the standardized picture, and final predictive classification determination is obtained by the trained neural network.

2. The deep learning-based quick and precise high-throughput drug screening system of claim 1, wherein said predictive classification determination is as follows:

| label | description |
|---|---|
| 0 | No efficacy |
| 1 | Low efficacy |
| 2 | Moderate efficacy |
| 3 | High efficacy. |

3. The deep learning-based quick and precise high-throughput drug screening system of claim 1, wherein the network structure of said neural network module is as follows:

| | convolution kernel (number) size/ |
|---|---|
| Types | stride (or notes) |
| convolution | (32) 3 × 3/1 |
| convolution | (64) 3 × 3/1 |
| convolution | (80) 1 × 1/1 |
| convolution | (192) 3 × 3/1 |
| pooling | (−) 3 × 3/2 |
| Module 1 | 3 × sub-network module 1 |
| Module 2 | 5 × sub-network module 2 |
| Module 3 | 3 × sub-network module 3 |
| pooling | (−) 8 × 8/1 |
| convolution | (4) 1 × 1/1 |
| Softmax | classification output. |

4. The deep learning-based quick and precise high-throughput drug screening system of claim 3, wherein the network structure of said sub-network module 1 is as follows:
input (Input goes into each branch)

| branch 1 | branch 2 | branch 3 | branch 4 |
|---|---|---|---|
| convolution | convolution | convolution | pooling (−) |
| (64) 1 × 1/1 | (48) 1 × 1/1 | (64) 1 × 1/1 | 3 × 3/1 |
| | | convolution | |
| | | (96) 3 × 3/1 | |
| | convolution | convolution | convolution |
| | (64) 5 × 5/1 | (96) 3 × 3/1 | (64) 1 × 1/1 |

Merge 4 branches along the channel direction.

5. The deep learning-based quick and precise high-throughput drug screening system of claim 3, wherein the network structure of said sub-network module 2 is as follows:
input (Input goes into each branch)

| branch 1 | branch 2 | branch 3 | branch 4 |
|---|---|---|---|
| convolution | convolution | convolution | pooling (−) |
| (192) 1 × 1/1 | (128) 1 × 1/1 | (128) 1 × 1/1 | 3 × 3/1 |
| | | convolution | |
| | | (128) 7 × 1/1 | |
| | convolution | convolution | |
| | (128) 1 × 7/1 | (128) 1 × 7/1 | |
| | | convolution | convolution |
| | | (128) 7 × 1/1 | (192) 1 × 1/1 |
| | convolution | convolution | |
| | (192) 7 × 1/1 | (192) 1 × 7/1 | |

Merge 4 branches along the channel direction.

6. The deep learning-based quick and precise high-throughput drug screening system of claim 3, wherein the network structure of said sub-network module 3 is as follows:
input (Input goes into each branch)

| branch 1 | branch 2 | | branch 3 | | branch 4 |
|---|---|---|---|---|---|
| convolution | convolution | | convolution | | pooling (−) |
| (320) 1 × 1/1 | (384) 1 × 1/1 | | (448) 1 × 1/1 | | 3 × 3/1 |
| | branch | branch | convolution | | |
| | 2a | 2b | (384) 3 × 3/1 | | |
| | convolution | convolution | branch | branch | |
| | (384) 1 × 3/1 | (384) 3 × 1/1 | 3a | 3b | |
| | | | |
|---|---|---|---|
| Merge 2 branches along the | convolution | convolution | convolution |
| channel direction | (384) 1 × 3/1 | (384) 3 × 1/1 | (192) 1 × 1/1 |

Merge 2 branches along the
channel direction
Merge 4 branches along the channel direction.

7. The deep learning-based quick and precise high-throughput drug screening system of claim 1, wherein the training method of neural network is as follows:
train neural network on two NVIDIA GTX 1080ti video cards by using TensorFlow framework; use Adam optimizer as training optimizer and determine the corresponding training parameters as: learning rate 0.001, beta1 0.9, beta2 0.999, epsilon 1e-8.

8. A method of deep learning-based quick and precise high-throughput drug screening, comprising:
1) treating A549 cells and HepG2 cells with traditional medicine and nano medicine for two hours and six hours respectively, then staining with fluorescent antibody and getting the cell picture;
2) inputting cell single-color channel pictures into a image preprocessing module to obtain standardized picture data;
3) importing standardized picture data into neural network module to obtain final predictive classification determination.

9. The method of claim 8, wherein said picture preprocessing module comprising a channel merging module and a picture standardization module; the input data of said channel merging module is cell single-color channel pictures, said channel merging module merges different cell single-color channel pictures into a multi-channel picture representation, and the tensor of the picture obtained after the merging is represented as [H,W,C]; said picture standardization module functions subsequent to said channel merging module, the input data of said picture standardization module is the tensor of merged picture, said picture standardization module standardizes the input data into the tensor representation of [70,70,C], the standardization method is as follows:
1) use bicubic interpolation algorithm to transform the picture tensor of [H, W, C] to [70, 70, C];
2) treat the picture tensor after interpolation by regularization method;
said neural network module functions subsequent to the picture standardization module, the input data of the neural network module is the tensor of the standardized picture, and final predictive classification determination is obtained by the trained neural network.

10. The method of claim 9, wherein said final predictive classification determination is as follows:

| label | description |
|---|---|
| 0 | No effcacy |
| 1 | Low effcacy |
| 2 | Moderate efficacy |
| 3 | High efficacy. |

11. The method of claim 9, wherein the network structure of said neural network module is as follows:

| | convolution kernel (number) size/ |
|---|---|
| Types | stride (or notes) |
| convolution | (32) 3 × 3/1 |
| convolution | (64) 3 × 3/1 |
| convolution | (80) 1 × 1/1 |
| convolution | (192) 3 × 3/1 |
| pooling | (−) 3 × 3/2 |
| Module 1 | 3 × sub-network module 1 |
| Module 2 | 5 × sub-network module 2 |
| Module 3 | 3 × sub-network module 3 |
| pooling | (−) 8 × 8/1 |
| convolution | (4) 1 × 1/1 |
| Softmax | classification output. |

12. The method of claim 11, wherein the network structure of said sub-network module 1 is as follows:
input (Input goes into each branch)

| branch 1 | branch 2 | branch 3 | branch 4 |
|---|---|---|---|
| convolution | convolution | convolution | pooling (−) |
| (64) 1 × 1/1 | (48) 1 × 1/1 | (64) 1 × 1/1 | 3 × 3/1 |
| | | convolution | |
| | | (96) 3 × 3/1 | |
| | convolution | convolution | convolution |
| | (64) 5 × 5/1 | (96) 3 × 3/1 | (64) 1 × 1/1 |

Merge 4 branches along the channel direction.

13. The method of claim 11, wherein the network structure of said sub-network module 2 is as follows:
input (Input goes into each branch)

| branch 1 | branch 2 | branch 3 | branch 4 |
|---|---|---|---|
| convolution | convolution | convolution | pooling (−) |
| (192) 1 × 1/1 | (128) 1 × 1/1 | (128) 1 × 1/1 | 3 × 3/1 |
| | | convolution | |
| | | (128) 7 × 1/1 | |
| | convolution | convolution | |
| | (128) 1 × 7/1 | (128) 1 × 7/1 | |
| | | convolution | convolution |
| | | (128) 7 × 1/1 | (192) 1 × 1/1 |
| | convolution | convolution | |
| | (192) 7 × 1/1 | (192) 1 × 7/1 | |

Merge 4 branches along the channel direction.

14. The method of claim 11, wherein the network structure of said sub-network module 3 is as follows:
input (Input goes into each branch)

| branch 1 | branch 2 | | branch 3 | | branch 4 |
|---|---|---|---|---|---|
| convolution | convolution | | convolution | | pooling (−) |
| (320) 1 × 1/1 | (384) 1 × 1/1 | | (448) 1 × 1/1 | | 3 × 3/1 |
| | branch | branch | convolution | | |
| | 2a | 2b | (384) 3 × 3/1 | | |
| | convolution | convolution | branch | branch | |
| | (384) 1 × 3/1 | (384) 3 × 1/1 | 3a | 3b | |
| | | | |
|---|---|---|---|
| Merge 2 branches along the | convolution | convolution | convolution |
| channel direction | (384) 1 × 3/1 | (384) 3 × 1/1 | (192) 1 × 1/1 |

Merge 2 branches along the
channel direction
Merge 4 branches along the channel direction.

15. The method of claim 11, wherein the training method of neural network is as follows: train neural network on two NVIDIA GTX 1080ti video cards by using TensorFlow framework; use Adam optimizer as training optimizer and determine the corresponding training parameters as: learning rate 0.001, beta1 0.9, beta2 0.999, epsilon 1e-8.
